(19) United States
(12) Patent Application Publication
(10) Pub. No.: US 2015/0327922 A1
(43) Pub. Date: Nov. 19, 2015

(54) MECHANICAL VIBRATIONS ON RF ABLATION DEVICES

(71) Applicant: Boston Scientific Scimed Inc., Maple Grove, MN (US)

(72) Inventors: Shuja Dawood, Cambridge, MA (US); Jennifer Leschkies, San Jose, CA (US); Anna Mandernach, Campbell, CA (US); Rebecca Tin, Sunnyvale, CA (US); Venkata Vegesna, Saratoga, CA (US); Paul A. Roche, San Jose, CA (US); Vikrant Singh Badwal, Sunnyvale, CA (US); Noel Keane, Campbell, CA (US); Adrian Valdelomar, (La Guacima) Alajuela (CR); Priya Sundaramurthy, Fremont, CA (US)

(21) Appl. No.: 14/712,470

(22) Filed: May 14, 2015

Related U.S. Application Data
(60) Provisional application No. 61/993,901, filed on May 15, 2014.

(51) Int. Cl. A61B 18/14

(52) U.S. Cl. CPC A61B 18/1492; A61B 2018/0019

(57) ABSTRACT

An ablation catheter system includes a tip assembly configured to provide ablation energy to tissue. The tip assembly includes an outer surface and a piezoelectric element is acoustically coupled to the outer surface of the tip assembly. The piezoelectric element is configured to cause the outer surface of the tip assembly to vibrate.

### CROSS-REFERENCE TO RELATED APPLICATION

This application claims priority to Provisional Application No. 61/993,901, filed May 15, 2014, which is herein incorporated by reference in its entirety.

### TECHNICAL FIELD

This application relates generally to medical devices and, more particularly, to catheters used to perform mapping and ablation functions.

### BACKGROUND

The treatment of cardiac arrhythmias is sometimes performed in conjunction with an ablation catheter inserted into a chamber of the heart or in one of the vessels leading into or from the heart. In the treatment of atrial fibrillation, for example, a radio frequency (RF) ablation catheter equipped with an electrode can be brought into contact with cardiac tissue for creating one or more ablation points along the tissue. During ablation, an RF generator supplies electrical energy to the electrode, generating an electric field in the tissue. The resulting resistive heat from this electric field forms a controlled lesion that blocks the electrical impulses from being conducted through the tissue and serves to promote the normal conduction of electrical impulses through the proper electrical pathway within the heart.

Often, during ablation, a thrombus may form on the ablation electrode tip of the catheter. Thrombus is an aggregation of proteins from the blood. One current technique for controlling or inhibiting thrombus formation in ablation procedures include utilizing an open-irrigated catheter design, whereby the ablation electrode is cooled, thereby regulating temperature and providing turbulent flow at the tip surface.

There is a continuing need for novel ablation catheters that exhibit reduced thrombus formation during ablation procedures.

### SUMMARY

Embodiments of the invention include an ablation catheter having a piezoelectric element that produces mechanical vibrations on the catheter's surface, thereby preventing thrombus proteins from readily attaching to the surface of the catheter. The mechanical vibrations may produce displacements, for example, on the micrometer scale and/or the nanometer scale.

According to Example 1, an ablation catheter system comprises a tip assembly configured to provide ablation energy to tissue, wherein the tip assembly includes an outer surface; and a piezoelectric element acoustically coupled to the tip assembly, wherein the piezoelectric element is configured to cause the outer surface of the tip assembly to vibrate.

In Example 2, the ablation catheter system according to Example 1, wherein the tip assembly further comprises a wall having an inside surface that defines a cavity, and wherein the system further comprises a portion of a steering mechanism coupled to the inside surface and extending proximally away from the tip assembly, wherein the piezoelectric element is disposed on the portion of the steering mechanism.

In Example 3, the ablation catheter system according to Example 2, wherein the portion of the steering mechanism includes a steering plate.

In Example 4, the ablation catheter system according to any of Examples 1 or 2, wherein the piezoelectric element is attached to a surface of the portion of the steering mechanism.

In Example 5, the ablation catheter system according to any of Examples 2-4, wherein the portion of the steering mechanism is laser-welded to the inside surface of the tip assembly.

In Example 6, the ablation catheter system according to any of Examples 1-5, wherein the piezoelectric element comprises a ring-shaped element.

In Example 7, the ablation catheter system according to any of Examples 1-6, wherein the piezoelectric element is attached to an interior surface of the tip assembly.

In Example 8, the ablation catheter system according to any of Examples 1 or 6, wherein the piezoelectric element is mechanically coupled to an outside surface of the tip assembly.

In Example 9, the ablation catheter system according to any of Examples 1-8, further comprising a power source coupled to the piezoelectric element, wherein the power source is configured to provide electrical power to the piezoelectric element.

In Example 10, the ablation catheter system according to Example 9, wherein the power source is further coupled to the tip assembly, and wherein the power source is further configured to provide electrical power to the tip assembly.

In Example 11, the ablation catheter system according to Example 9, further comprising an additional power source coupled to the tip assembly, and wherein the additional power source is configured to provide electrical power to the tip assembly.

In Example 12, the ablation catheter system according to any of Examples 1-11, wherein the piezoelectric element is configured to vibrate at a frequency that is greater than about one megahertz.

In Example 13, the ablation catheter system according to any of Examples 1-12, wherein the piezoelectric element is configured to cause at least one of microvibrations and nanovibrations.

In Example 14, a method comprises providing an ablation catheter having a tip assembly and a piezoelectric element, wherein the tip assembly is configured to deliver radio frequency (RF) ablation energy and includes an outer surface, and the piezoelectric element is coupled to the tip assembly; and supplying electrical energy to the piezoelectric element so as to cause the piezoelectric element and the outer surface of the tip assembly to vibrate.

In Example 15, the method according to Example 14, wherein the ablation catheter further comprises one or more microelectrodes coupled to a mapping signal processor, wherein the one or more microelectrodes and the mapping signal processor are configured to produce electrocardiogram signals during an ablation procedure, and wherein the method further comprises filtering frequencies corresponding to vibrations of the piezoelectric element from the electrocardiogram signals using the mapping signal processor.

In Example 16, an ablation catheter system comprises a tip assembly configured to provide ablation energy to tissue, wherein the tip assembly includes an outer surface; and a piezoelectric element acoustically coupled to the tip assembly, wherein the piezoelectric element is configured to cause the outer surface of the tip assembly to vibrate.

In Example 17, the ablation catheter system according to Example 16, wherein the tip assembly further comprises a wall having an inside surface that defines a cavity, and wherein the system further comprises a portion of a steering mechanism coupled to the inside surface and extending proximally away from the tip assembly, wherein the piezoelectric element is disposed on the portion of the steering mechanism.

In Example 18, the ablation catheter system according to Example 17, wherein the portion of the steering mechanism includes a steering plate.

In Example 19, the ablation catheter system according to Example 18, wherein the piezoelectric element is attached to a surface of the portion of the steering plate.

In Example 20, the ablation catheter system according to any of Examples 17-19, wherein the portion of the steering mechanism is laser-welded to the inside surface of the tip assembly.

In Example 21, the ablation catheter system according to any of Examples 16-20, wherein the piezoelectric element comprises a ring-shaped element.

In Example 22, the ablation catheter system according to Example 21, wherein the piezoelectric element is attached to an interior surface of the tip assembly.

In Example 23, the ablation catheter system according to Example 21, wherein the piezoelectric element is mechanically coupled to an outside surface of the tip assembly.

In Example 24, the ablation catheter system according to any of Examples 16-23, further comprising a power source coupled to the piezoelectric element, wherein the power source is configured to provide electrical power to the piezoelectric element.

In Example 25, the ablation catheter system according to Example 24, wherein the power source is further coupled to the tip assembly, and wherein the power source is further configured to provide electrical power to the tip assembly.

In Example 26, the ablation catheter system according to Example 24, further comprising an additional power source coupled to the tip assembly, and wherein the additional power source is configured to provide electrical power to the tip assembly.

In Example 27, the ablation catheter system according to any of Examples 16-26, wherein the piezoelectric element is configured to vibrate at a frequency that is greater than about one megahertz.

In Example 28, the ablation catheter system according to any of Examples 16-27, wherein the piezoelectric element is configured to cause at least one of microvibrations and nanovibrations.

In Example 29, the ablation catheter according to any of Examples 16-28, further comprising one or more microelectrodes coupled to a mapping signal processor, wherein the one or more microelectrodes and the mapping signal processor are configured to produce electrocardiogram signals during an ablation procedure, and wherein the piezoelectric element is configured to vibrate at a frequency that is filtered by the mapping signal processor.

In Example 30, an ablation catheter system comprises a tip assembly comprising a means for providing ablation energy to tissue; and a means for causing an outer surface of the tip assembly to vibrate, said means including a piezoelectric element.

In Example 31, the ablation catheter system according to Example 30, wherein the piezoelectric element is configured to vibrate at a frequency that is greater than about one megahertz.

In Example 32, the ablation catheter system according to Example 30 or 31, wherein the piezoelectric element is configured to cause at least one of microvibrations and nanovibrations.

In Example 33, the ablation catheter system according to any of Examples 30-32, wherein the means for providing ablation energy to tissue comprises a distal portion of the tip assembly, the distal portion of the tip assembly having an outer surface, and wherein the piezoelectric element is acoustically coupled to the outer surface.

In Example 34, the ablation catheter system according to any of Examples 30-33, wherein the piezoelectric element comprises a ring element mechanically coupled to the outer surface.

In Example 35, the ablation catheter system according to any of Examples 30-34, the means for causing an outer surface of tip assembly to vibrate further comprising a power source coupled to the piezoelectric element, wherein the power source is configured to provide electrical power to the piezoelectric element, and wherein the power source is further configured to provide electrical power to the tip assembly.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram depicting an ablation catheter system in accordance with embodiments of the invention;

FIG. 2 is a cross-section side view of a distal portion of an ablation catheter in accordance with embodiments of the invention;

FIG. 3 is a cross-section perspective view of the steering mechanism of the ablation catheter depicted in FIG. 2, in accordance with embodiments of the invention;

FIG. 4 is a cut-away perspective view of an ablation catheter in accordance with embodiments of the invention; and

FIG. 5 is a cut-away perspective view of an ablation catheter in accordance with embodiments of the invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

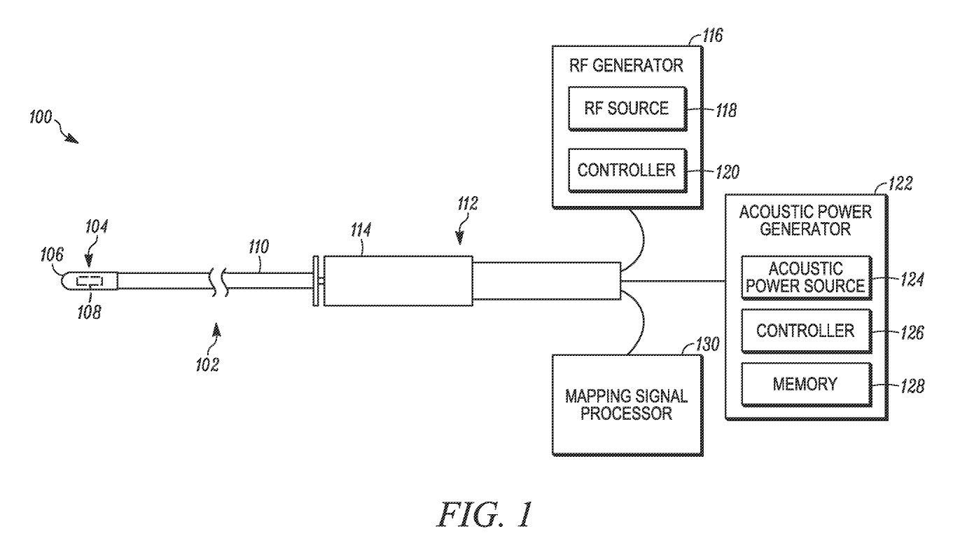

FIG. 1 is a schematic view illustrating embodiments of a mapping and ablation system 100 that includes a catheter 102. In the illustrated embodiments, the catheter 102 may be a hybrid catheter that can be used simultaneously for both localized mapping and ablation functions. That is, for example, the hybrid catheter 102 may be configured to provide localized, high resolution ECG signals during ablation. In other embodiments, the catheter may not include mapping functions. The illustrated catheter 102 includes an ablation tip assembly 104 having a piezoelectric element 108 disposed within, on, or otherwise connected to, the ablation tip assembly 104. The tip assembly 104 is coupled to a distal end of a catheter body 110 and a proximal catheter handle assembly 112, having a handle 114. The catheter body 110 includes one or more lumens (e.g., tubular elements) to provide conduits for components such as electrical conductors, irrigation/cooling fluid, a thermocouple or thermistor, an insertable stylet, a steering mechanism, a positioning system, and/or the like. In some embodiments, the catheter 102 may be an open, closed, or non-irrigated catheter design.

The catheter body 110 may be flexible to allow the catheter 102 to be steered through the vasculature of the patient. A steering wire (not shown) may be slidably disposed within the catheter body 110. The handle assembly 112 may include a steering member such as a slider, lever mechanism, or rotating steering knob (not shown) that is mounted to the handle 114. Actuation of the steering wire may be accomplished, for example, through rotational and/or translational movement at the handle 114. Rotational or translational movement of the handle actuation mechanism relative to the handle 114 in a first direction may cause a steering wire to move proximally relative to the main body 110 which, in turn, tensions the steering wire, thus pulling and bending the catheter body 110 into an arc; and returning the handle actuation mechanism to its original position on the handle 114 may cause the steering wire to move distally relative to the catheter body 110 which, in turn, relaxes the steering wire, thus allowing the catheter to return toward its form.

The illustrated system 100 includes an RF generator 116 used to generate energy for ablation procedures. The RF generator 116 includes an RF energy source 118 and a controller 120 for controlling, for example, the timing and the level of the RF energy delivered through the tip assembly 104. During an ablation procedure, the RF generator 116 may be configured to deliver ablation energy to the tip assembly 104 in a controlled manner to ablate sites identified or targeted for ablation. Other types of ablation sources in addition to, or in lieu of, the RF generator 116 can also be used for ablating target sites. Examples of other types of ablation sources can include, but are not limited to, microwave generators, acoustic generators, cryoablation fluid sources, and laser/optical generators.

The illustrated system 100 includes an acoustic power generator 122 for powering the piezoelectric element 108. The acoustic power generator 122 may provide a steady power source, an adjustable power source, and/or the like. In embodiments, the acoustic power generator 122 may be an independent component, as shown in FIG. 1. In other embodiments, the acoustic power generator 122 may be integrated into the RF generator 116. That is, for example, the RF source 118 may provide power for generating energy for ablation and for powering the piezoelectric element 108. In embodiments, the RF circuitry may be bifurcated within the handle assembly 112, with a first portion of the power being directed to the tip assembly 104, and a second portion of power being directed to the piezoelectric element 108. In these embodiments, the power for the piezoelectric element 108 may undergo frequency conversion in the handle, e.g., from a supplied 460 Hz to an operating frequency of the piezoelectric element 108. The frequency conversion may be accomplished using a frequency multiplier circuit and/or other frequency modification mechanisms.

In the illustrated embodiment, the acoustic power generator 122 includes an acoustic power source 124 and a controller 126. The acoustic power source 124 may include, for example, one or more batteries, one or more capacitors, and/or power circuits configured to supply power to the piezoelectric element 108, e.g., using capacitive and/or magnetic coupling. In embodiments, for example, the acoustic power source 124 may include a capacitor that draws power from the RF source 118. The controller 126 may be configured to cause power to be provided to the piezoelectric element 108 from the acoustic power source 124. In embodiments, for example, the controller 126 may be configured to cause the piezoelectric element 108 to vibrate with particular amplitudes, frequencies, bursts, phases, patterns, and/or the like.

Electrical signals, such as electrocardiograms (ECGs), may be used during a cardiac ablation procedure to distinguish viable tissue from not viable tissue. If ECG amplitudes are seen to attenuate during the delivery of RF energy into the tissue, the delivery of RF energy into that specific tissue may be stopped. However, noise in the ECG signals may make it difficult to view attenuation. For example, mechanical vibrations having frequencies that are near the frequencies of the ECG signals can cause noise in the ECG signals. Accordingly, in embodiments, the controller 126 may be configured to cause the piezoelectric element 108 to vibrate with a frequency that is different from (e.g., greater than) the frequency being used for ECG signals.

In embodiments, the controller 126 may be, or include, controller 120. The controller 126 may be, or include, one or more circuits, one or more programmable microcontrollers or microprocessors, one or more programmable logic devices (PLDs), one or more application specific integrated circuits (ASICs), memory, hardware, software, firmware and/or the like. The controller 126 may execute instructions and perform desired tasks as specified by the instructions. The controller 126 may also be configured to store information in a memory 128 and/or access information from the memory 128. The memory 128 may include volatile and/or non-volatile memory, and may store instructions that, when executed by the controller 128 cause methods and processes to be performed by the catheter 102. For example, in embodiments, the controller 126 may process instructions and/or data stored in the memory 128 to control delivery of ablation energy by the catheter 102. Although the present system is described in conjunction with an ablation catheter system 100 having a microprocessor-based architecture, it will be understood that the catheter system 100 (or other device) may be implemented in any logic-based integrated circuit architecture, if desired.

A mapping signal processor 130 is connected to electrodes (not shown in FIG. 1). The mapping signal processor 130 and electrodes detect electrical activity of the heart. This electrical activity is evaluated to analyze an arrhythmia and to determine where to deliver the ablation energy as a therapy for the arrhythmia. One of ordinary skill in the art will understand that, the components and other circuitry shown and described herein can be implemented using software, hardware, and/or firmware. Various disclosed methods may be implemented as a set of instructions contained on a computer-accessible medium capable of directing a processor to perform the respective method.

The illustrative system 100 shown in FIG. 1 is not intended to suggest any limitation as to the scope of use or functionality of embodiments of the invention disclosed throughout this document. Neither should the illustrative system 100 be interpreted as having any dependency or requirement related to any single component or combination of components illustrated therein. For example, in embodiments, the illustrative system 100 may include additional components such as, for example, a sensor circuit (not illustrated). Additionally, any one or more of the components depicted in FIG. 1 can be, in embodiments, integrated with various ones of the other components depicted therein (and/or components not illustrated). For example, the controller 120 and the controller 126 may be, or include, one integrated controller. Any number of other components or combinations of components can be integrated with the illustrative system 100 depicted in FIG. 1, all of which are considered to be within the ambit of the invention.

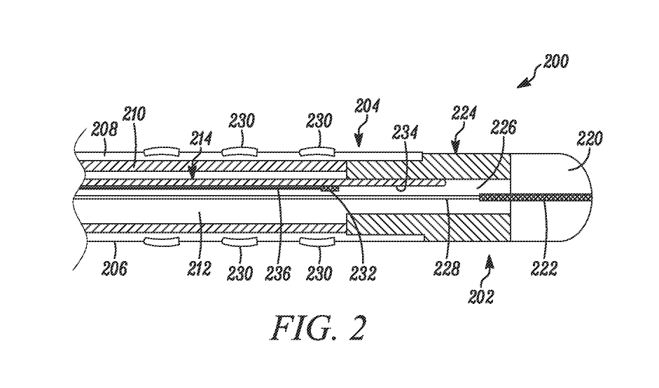

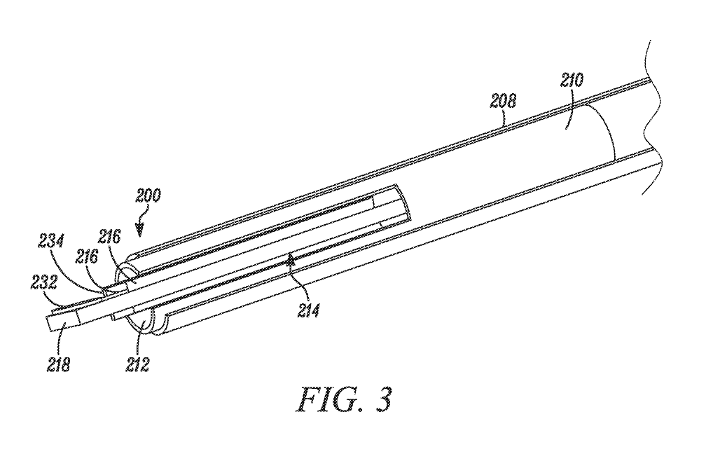

FIG. 2 is a cross-section side view of a portion of an ablation catheter 200 in accordance with embodiments of the invention, and FIG. 3 is a cross-sectional perspective view of a distal portion of the ablation catheter 200 according to an embodiment. In various embodiments, catheter 200 may be similar to, or include, the catheter 102 depicted in FIG. 1. As shown in FIG. 2, the ablation catheter 200 includes an ablation tip assembly 202 coupled to a distal end 204 of a catheter body 206. In some embodiments, the catheter body 206 comprises a layer 208 of flexible tubing (e.g., plastic tubing) disposed over a layer 210 to increase the rotational stiffness of the body. In embodiments, the layer 210 may include a braided mesh, one or more coils, a reinforcement sleeve, and/or the like. As shown in FIG. 3, the catheter body 206 includes at least one lumen 212 defined therein. As illustrated, a steering mechanism 214 is disposed within the lumen 212 and may be configured to facilitate steering (e.g., controlling) movement of the catheter 200 inside of a patient's body. As shown, the steering mechanism 214 may include a number of steering wires 216 coupled to a support plate 218. In embodiments, the steering mechanism 214 may include a single steering wire coupled to a support plate, just a steering wire, just a support plate, and/or some combination of these and/or other elements. The skilled artisan will recognize that the particular steering mechanism 214 illustrated in FIGS. 2 and 3 is illustrative only, and that any number of steering/deflection arrangements may be utilized within the scope of the various embodiments.

As shown in FIG. 2, the ablation tip assembly 202 includes a distal tip 220. A temperature sensor 222 such as a thermocouple or thermistor may be at least partially disposed within the distal tip 220. As shown in FIG. 2, the ablation tip assembly 202 also includes a proximal portion 224 within which is defined a cavity 226. As shown, the distal tip 220 is disposed distally of the proximal portion 224. In various embodiments, a temperature sensor wire 228 extends proximally from the sensor 222. The tip assembly 202, or some portion thereof, may be formed from a conductive material. For example, some embodiments, the distal tip 220 is formed from a conductive material such as a platinum alloy. This conductive material may be used, for example, to conduct RF energy used to form lesions in targeted tissue during the ablation procedure. In this manner, the distal tip 220 serves as an RF ablation electrode. The specific configuration of the distal tip 220 can vary among the various embodiments, depending on the particular clinical needs of the patient. According to embodiments, the tip assembly 202 may include a distal tip 220 having a length of approximately 4-4.5 millimeters. In other embodiments, the distal tip 220 may have a length of approximately 8 millimeters. In other embodiments, the distal tip 220 may have different lengths.

In embodiments, one or more mapping electrodes 230 may be included within, or disposed on, the tip assembly 202. In embodiments, mapping electrodes 230 may also, or alternatively, be included within, or disposed on, the catheter body 206. The mapping electrodes 230 may be used for a mapping function to sense intrinsic intra cardiac activity.

In various embodiments, the tip assembly 202 may include one or more high-resolution microelectrodes configured to sense high resolution, precise localized electrical activity, which may be used, for example, to assess ablation lesion formation, control the temperature of the distal tip 220 (serving as the RF ablation electrode), to assess the formation of coagulum proximate the distal tip 220, and/or to provide the ability to diagnose complex ECG activity.

According to embodiments, a piezoelectric element 232 may be fixedly attached to a portion of the steering mechanism 214. For example, in various embodiments, the piezoelectric element 232 may be attached to steering wire 216 or the support plate 218 of the steering mechanism 214. In the particular embodiment illustrated in FIGS. 2, 3, for example, the piezoelectric element 232 may be disposed on a lower surface 234 of the support plate 218 and power may be supplied thereto by a wire 236. Attaching the piezoelectric element 232 to the support plate 218, which in turn is attached to an inner surface of the distal tip 220, results in vibrations emitted in the piezoelectric element 232 being transmitted to the outer surface of the distal tip 220. The corresponding high frequency vibrations of the distal tip 220 will operate to inhibit the attachment of RBCs to the distal tip 220, which in turn will inhibit thrombus formation proximate the distal tip 220.

In embodiments, vibrational loss to connecting materials (e.g., solder) may be minimized by laser-welding the support plate 218 to an inner surface of the distal tip 220. In other embodiments, other techniques, e.g., brazing, soldering, press-fits, mechanical couplings, adhesive joints, resistance welding, laser welding, and the like may be utilized to attach the support plate 218 to the tip assembly 202.

In embodiments, the catheter 200 may be configured to use off-the shelf piezoelectric elements (e.g., as custom-designed piezoelectric elements may not be necessary), custom-designed piezoelectric elements, and/or the like. In various embodiments, the number and placement of piezoelectric elements 232 may be determined based on any number of criteria such as, for example, to maximize vibrational efficiency, to achieve a desired frequency or frequencies, to minimize vibrational loss, and/or the like.

According to embodiments, the vibrations of the piezoelectric element 232 may be in the megahertz range so as to minimize interference with the RF treatment (e.g., due to the disparity in wavelengths). For example, the piezoelectric element 232 may be configured to vibrate about or above 1 MHz where the RF signal has a frequency of about 460 kHz. The piezoelectric element 232 may be configured to vibrate at times when RF power is not being actively delivered by the RF electrode (e.g., tip assembly 202) such as, for example, by configuring alternating timing cycles for the piezoelectric element 232 and the RF generator (e.g., the RF generator 116 depicted in FIG. 1). Additionally, the catheter 200 may be configured such that the vibrations produced by the piezoelectric element 232 resonate at a frequency high enough to get filtered by recording systems.

The illustrative ablation catheter 200 shown in FIGS. 2 and 3 is not intended to suggest any limitation as to the scope of use or functionality of embodiments of the invention disclosed throughout this document. Neither should the illustrative ablation catheter 200 be interpreted as having any dependency or requirement related to any single component or combination of components illustrated therein. For example, in embodiments, the illustrative ablation catheter 200 may include additional components such as, for example, additional piezoelectric elements. Additionally, any one or more of the components depicted in FIGS. 2 and 3 can be, in embodiments, integrated with various ones of the other components depicted therein (and/or components not illustrated). Any number of other components or combinations of components can be integrated with the illustrative ablation catheter 200 depicted in FIGS. 2 and 3, all of which are considered to be within the ambit of the invention.

As described above, embodiments of the invention include an ablation catheter having a tip assembly to which mechanical vibrations are imparted to prevent thrombus generation. According to embodiments, the mechanical vibrations are caused by one or more piezoelectric elements acoustically coupled to the ablation tip assembly. For example, a piezoelectric element may be disposed on a steering mechanism element such as a steering wire or support plate. One or more piezoelectric elements (e.g., ring elements) may be disposed on and/or adjacent to the tip assembly. According to embodiments, for example, use of a ring piezoelectric element facilitates mechanical attachment of the piezoelectric element directly to the tip assembly.

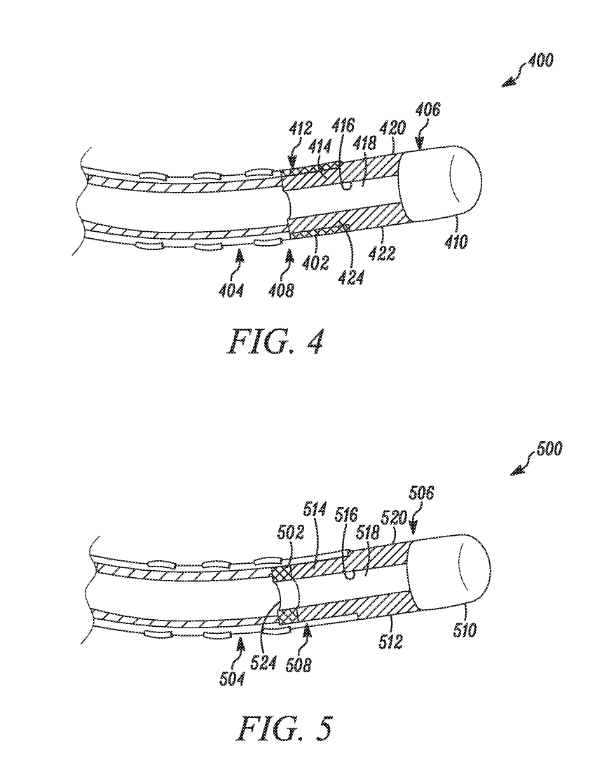

FIG. 4 is a cut-away perspective view of an ablation catheter 400 that includes a ring-shaped piezoelectric element 402, in accordance with embodiments of the invention. As shown, the catheter 400 includes a catheter body 404 and a tip assembly 406 coupled to a distal end 408 of the catheter body 404. As shown, the tip assembly 406 includes a distal tip 410 and a proximal portion 412. In various embodiments, the distal tip 410 can operate as an RF ablation electrode. The proximal portion 412 includes a generally cylindrical wall 414 having an inner surface 416 that defines an internal lumen 418. The wall 414 also includes an outer surface 420. As shown, in the particular embodiment illustrated, a distal portion 422 of the wall 414 is thicker than a proximal portion 424 of the wall 414. In the illustrated embodiment, the piezoelectric element 402 is disposed on the outer surface 420 of the proximal portion 424 of the wall 414. The piezoelectric element 402 may be fixedly attached to the outer surface 420 so that vibrations produced by the piezoelectric element 402 are transferred directly to the tip assembly 406. In embodiments, more than one piezoelectric element 402 may be used. In embodiments in which the piezoelectric element 402 is located on an external surface of the tip assembly 406, piezo materials may be configured to have characteristics such as, for example, thermal expansion, certain operating temperature ranges, biocompatibility, and/or the like.

FIG. 5 is a cut-away perspective view of an ablation catheter 500 that includes a ring-shaped piezoelectric element 502, in accordance with embodiments of the invention. As shown, the catheter 500 includes a catheter body 504 and a tip assembly 506 coupled to a distal end 508 of the catheter body 504. As shown, the tip assembly 506 includes a distal tip 510 and a proximal portion 512. In various embodiments, the distal tip 510 can operate as an RF ablation electrode. The proximal portion 512 includes a generally cylindrical wall 514 having an inner surface 516 that defines an internal lumen 518. The wall 514 also includes an outer surface 520. In the illustrated embodiment, a piezoelectric element 502 is coupled to a proximal end 524 of the proximal portion 512 of the tip assembly 506. The piezoelectric element 502 may be fixedly attached to the proximal end 524 of the proximal portion 512 of the tip assembly 506. In this manner, vibrations produced by the piezoelectric element 502 are transferred directly to the tip assembly 506. In embodiments, more than one piezoelectric element 502 may be used. According to embodiments, a piezoelectric element 502 may be disposed in the lumen 518 and may, for example, extend at least a portion of the length of the lumen 518 of the tip assembly 506. In embodiments, a small cavity may be included behind the piezoelectric element 502 to receive wires.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An ablation catheter system, the system comprising:
a tip assembly configured to provide ablation energy to tissue, wherein the tip assembly includes an outer surface; and
a piezoelectric element acoustically coupled to the tip assembly, wherein the piezoelectric element is configured to cause the outer surface of the tip assembly to vibrate.

2. The ablation catheter system of claim 1, wherein the tip assembly further comprises a wall having an inside surface that defines a cavity, and wherein the system further comprises a portion of a steering mechanism coupled to the inside surface and extending proximally away from the tip assembly, wherein the piezoelectric element is disposed on the portion of the steering mechanism.

3. The ablation catheter system of claim 2, wherein the portion of the steering mechanism includes a steering plate.

4. The ablation catheter system of claim 3, wherein the piezoelectric element is attached to a surface of the portion of the steering plate.

5. The ablation catheter system of claim 1, wherein the portion of the steering mechanism is laser-welded to the inside surface of the tip assembly.

6. The ablation catheter system of claim 1, wherein the piezoelectric element comprises a ring-shaped element.

7. The ablation catheter system of claim 6, wherein the piezoelectric element is attached to an interior surface of the tip assembly.

8. The ablation catheter system of claim 6, wherein the piezoelectric element is mechanically coupled to an outside surface of the tip assembly.

9. The ablation catheter system of claim 1, further comprising a power source coupled to the piezoelectric element, wherein the power source is configured to provide electrical power to the piezoelectric element.

10. The ablation catheter system of claim 9, wherein the power source is further coupled to the tip assembly, and wherein the power source is further configured to provide electrical power to the tip assembly.

11. The ablation catheter system of claim 9, further comprising an additional power source coupled to the tip assembly, and wherein the additional power source is configured to provide electrical power to the tip assembly.

12. The ablation catheter system of claim 1, wherein the piezoelectric element is configured to vibrate at a frequency that is greater than about one megahertz.

13. The ablation catheter system of claim 1, wherein the piezoelectric element is configured to cause at least one of microvibrations and nanovibrations.

14. The ablation catheter of claim 1, further comprising one or more microelectrodes coupled to a mapping signal processor, wherein the one or more microelectrodes and the mapping signal processor are configured to produce electrocardiogram signals during an ablation procedure, and wherein the piezoelectric element is configured to vibrate at a frequency that is filtered by the mapping signal processor.

15. An ablation catheter system, comprising:
a tip assembly comprising a means for providing ablation energy to tissue; and
a means for causing an outer surface of the tip assembly to vibrate, said means including a piezoelectric element.

16. The ablation catheter system of claim 15, wherein the piezoelectric element is configured to vibrate at a frequency that is greater than about one megahertz.

17. The ablation catheter system of claim 15, wherein the piezoelectric element is configured to cause at least one of microvibrations and nanovibrations.

18. The ablation catheter system of claim 15, wherein the means for providing ablation energy to tissue comprises a distal portion of the tip assembly, the distal portion of the tip assembly having an outer surface, and wherein the piezoelectric element is acoustically coupled to the outer surface.

19. The ablation catheter system of claim 18, wherein the piezoelectric element comprises a ring element mechanically coupled to the outer surface.

20. The ablation catheter system of claim 15, the means for causing an outer surface of tip assembly to vibrate further comprising a power source coupled to the piezoelectric element, wherein the power source is configured to provide electrical power to the piezoelectric element, and wherein the power source is further configured to provide electrical power to the tip assembly.
